(19) United States
(12) Patent Application Publication
(10) Pub. No.: US 2025/0049802 A1
(43) Pub. Date: Feb. 13, 2025

(54) COMBINATION THERAPIES AND USES FOR TREATING CANCER

(71) Applicant: Pfizer Inc., New York, NY (US)

(72) Inventors: Stephen George DANN, Poway, CA (US); Koleen Jill EISELE, San Diego, CA (US); Conglin FAN, San Diego, CA (US); Manfred KRAUS, San Diego, CA (US); Pei-Pei KUNG, San Diego, CA (US); Shikhar SHARMA, San Diego, CA (US); Hui WANG, San Diego, CA (US)

(73) Assignee: Pfizer Inc., New York, NY (US)

(21) Appl. No.: 18/719,456

(22) PCT Filed: Dec. 12, 2022
(86) PCT No.: PCT/IB2022/062065

§ 371 Date: Jun. 13, 2024

Related U.S. Application Data
(60) Provisional application No. 63/289,180, filed on Dec. 14, 2021.

(51) Int. Cl. A61K 31/519; A61K 31/444; A61K 31/567; A61P 35/00

(52) U.S. Cl. CPC A61K 31/519; A61K 31/444; A61K 31/567; A61P 35/00

(57) ABSTRACT

The present disclosure relates to the treatment of cancer and/or cancer-associated disease. Certain aspects relate to the treatment of an individual having cancer or cancer-associated disease by administering to the individual a combination therapy of a first therapeutic agent that is an EZH2 inhibitor, a second therapeutic agent that is a CDK inhibitor, and a third therapeutic agent that is a selective estrogen receptor degrader.

### FIELD OF THE INVENTION

The present invention relates to combination therapies useful for the treatment of cancer and/or cancer-associated diseases. In particular, the invention relates to a combination therapy which comprises an enhancer of zeste homolog 2 (EZH2) inhibitor or a pharmaceutically acceptable salt thereof, a cyclin-dependent kinase (CDK) inhibitor or a pharmaceutically acceptable salt thereof, and a selective estrogen receptor degrader (SERD) or a pharmaceutically acceptable salt thereof. The invention also relates to associated methods of treatment, pharmaceutical combinations, and pharmaceutical uses.

### BACKGROUND

Breast cancer is one of the most common malignant tumors in women. Breast cancer is generally categorized into three major subtypes based on the presence or absence of molecular markers for estrogen or progesterone receptors (HR) and human epidermal growth factor 2 (HER2): hormone receptor positive and HER2 negative (70% of patients), HER2 positive (15%-20%), and triple-negative (tumors lacking all 3 standard molecular markers; 15%).

Accordingly, there is a pressing need for clinically active agents and improved therapies for treatment of hormone receptor positive and HER2 negative (HR+/HER2−) locally advanced unresectable or metastatic breast cancer, and/or cancer-associated diseases.

Treatment options for cancer patients have been contemplated in the art. For example, WO 2021/063322 A1 has disclosed a single example of a dual-combination therapy with an EZH2 inhibitor and a CDK4/6 inhibitor. However, there remains a need for more effective and safe therapeutic agents and a need for their preferential use in combination therapy. For example, there is a need for a combination therapy that can control the tumor sizes for a longer period before it is relapsed and is less toxic to patients. Preferred combination therapies of the present invention show greater efficacy and better safety profile than treatment with either therapeutic agent alone.

### SUMMARY

This invention relates to therapies, including combination therapies for the treatment of cancer and/or cancer-associated diseases. Provided herein are methods of treating a cancer and/or a cancer-associated disease in a subject. Also provided are methods of inhibiting tumor growth or progression in a subject who has malignant cells. Also provided are methods of inhibiting metastasis of malignant cells in a subject. Also provided are methods of inducing tumor regression in a subject who has malignant cells.

Disclosed herein are methods of treating a cancer and/or a cancer-associated disease in a subject comprising administering to the subject a combination therapy which comprises a first therapeutic agent that is an EZH2 inhibitor or a pharmaceutically acceptable salt thereof, a second therapeutic agent that is a CDK inhibitor or a pharmaceutically acceptable salt thereof, and a third therapeutic agent that is fulvestrant or a pharmaceutically acceptable salt thereof. Preferably, the method of the present invention comprises administering to the subject a combination therapy which comprises a first therapeutic agent that is an EZH2 inhibitor or a pharmaceutically acceptable salt thereof, a second therapeutic agent that is a CDK inhibitor or a pharmaceutically acceptable salt thereof, and a third therapeutic agent that is fulvestrant or a pharmaceutically acceptable salt thereof.

EZH2 inhibitor of this invention may be but is not limited to CPI-1205, GSK126, valemetostat, tazemetostat, PF-06821497, GSK-2816126, 3-deazaneplanocin A, or a pharmaceutically acceptable salt thereof.

Preferably, the EZH2 inhibitor is PF-06821497, which is a compound of Formula A:

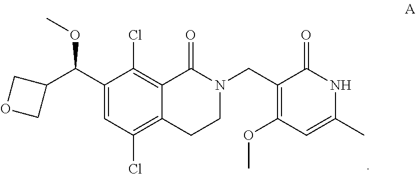 [SMILES: COc1cc(C)[nH]c(=O)c1CN1CCc2c(Cl)cc([C@H](OC)C3COC3)c(Cl)c2C1=O]

Preferably, the CDK inhibitor is palbociclib having Formula B:

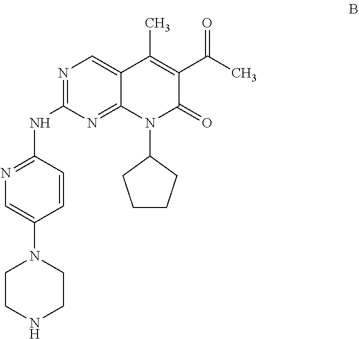 [SMILES: CC(=O)c1c(C)c2cnc(Nc3ccc(N4CCNCC4)cn3)nc2n(C2CCCC2)c1=O]

or a pharmaceutically acceptable salt thereof.

More preferably, the CDK inhibitor is PF-06873600 having Formula C:

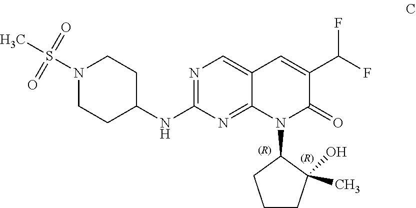

or a pharmaceutically acceptable salt thereof.

Fulvestrant is a compound of Formula D:

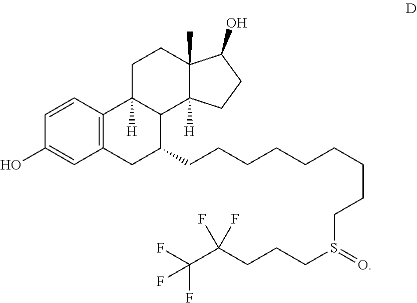

In some aspects, at least one of the therapeutic agents is administered to a subject in an intravenous (IV), subcutaneous (SC), or oral dose (PO).

In some aspects, the cancer being treated is breast cancer.

In some aspects, the cancer being treated is hormone receptor positive and HER2 negative (HR⁺/HER2⁻) breast cancer.

### BRIEF DESCRIPTION OF THE FIGURES

FIG. 1 depicts the efficacy evaluation of monotherapy treatment of PF-06821497, dual combination treatment with palbociclib and fulvestrant, and triple combination treatment with PF-06821497, palbociclib and fulvestrant in HCC1428 model.

FIG. 2 depicts the efficacy evaluation of monotherapy treatment of PF-06821497, dual combination treatment with PF-06873600 and fulvestrant, and triple combination treatment with PF-06821497, PF-06873600 and fulvestrant in HCC1428 model.

FIG. 3 depicts the body weight change during PF-06821497 single or combination treatment in the HCC1428 model.

FIG. 4 depicts the efficacy evaluation of PF-06821497 single or combination treatment with palbociclib and fulvestrant in the ST941 PDX model.

FIG. 5 depicts the body weight change during PF-06821497 single or combination treatment with palbociclib and fulvestrant in the ST941 PDX model.

### DETAILED DESCRIPTION

The instant application relates to the treatment of cancer and/or cancer-associated disease. Certain aspects relate to the treatment of an individual having cancer or cancer-associated disease by administering to the individual a combination therapy of a first therapeutic agent that is an EZH2 inhibitor, a second therapeutic agent that is a CDK inhibitor, and a third therapeutic agent that is fulvestrant.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The terms “cancer”, “cancerous”, or “malignant” refer to or describe the physiological condition in mammals that is typically characterized by unregulated cell growth.

“Therapeutic agent” is a chemical compound useful in the treatment of cancer and/or cancer-associated disease.

“Therapy” as used herein, refers to a therapeutic agent, as defined above. “Combination therapy” as used herein, refers to a combination of two, or three therapeutic agents, for the treatment of cancer and/or cancer-associated disease. When a therapy has more than one therapeutic agent, the therapeutic agents can be part of the same or separate dosages forms and are administered to the patient via the same or different route of administration and on the same or different administration schedules.

“Patient”, “subject” or “individual” refers to any living organism suffering from or prone to a condition that can be prevented or treated by administration of a therapeutic agent or composition or combination as provided herein, such as a cancer and/or a cancer-associated disease. “Patient”, “subject” or “individual” may include both humans and animals.

As used in herein, “administering” refers to the delivery of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion.

“Treat” or “treating” a cancer and/or a cancer-associated disease as used herein means to administer a combination therapy according to the present invention to a subject, patient or individual having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The terms “treatment regimen”, “dosing protocol” and “dosing regimen” are used interchangeably to refer to the dose and timing of administration of each therapeutic agent in a combination of the invention.

As used herein, an “effective dosage”, “effective amount”, or “therapeutically effective amount” of drug, compound, or pharmaceutical composition is an amount sufficient to affect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein “dosing” refers to both the “dose amount”, for example 1 mg, 20 mg, and the “dose frequency”, for example, once a day (QD), once a week (Q1W or QW), every two weeks (Q2W), every three weeks (Q3W) and every four weeks (Q4W). Dosing may also include the administration route of a drug, such as for example, subcutaneously (SC), intravenously (IV), oral (PO), if so specified. Similarly, a “priming dosing”, a “first treatment dosing”, a “second treatment dosing” and so on, each refers to both the dose amount and dose frequency of such dosing and optionally also includes the administration route if so specified. In some embodiments, there are one dose amount and one dose frequency in a dosing. In some embodiments, there are more than one dose amounts, and/or more than one dose frequencies in a dosing.

As used herein, “cycle”, and “week” when used in the context of describing a method of treating cancer including uses thereof, a dosing, or a dosing schedule, refer to a duration of time. A cycle is 21 days or 28 days, unless otherwise specified, when a subject is treated with a therapeutic agent, a pharmaceutical product thereof, or any pharmaceutical product thereof, as a monotherapy or in combination with a second therapeutic agent. Week 1 refers to the first week when the subject is treated under the method, or any of the dosing or dosing schedules therein unless otherwise specified. Week 2 starts immediately after week 1 ends, week 3 starts immediately after week 2 ends, and so on. Cycle 1 starts on the first day of week 1, the first day of week 2, or the first day of week three, unless otherwise specified. Unless stated otherwise, cycle 2 starts immediately after cycle 1 ends, cycle 3 starts immediately after cycle 2 ends, and so on.

“Tumor” as it applies to a subject diagnosed with, or suspected of having, a cancer refers to a malignant or potentially malignant neoplasm or tissue mass of any size. “Tumor” may include primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms). Multiple myeloma is a cancer of the plasma cells

“Tumor burden” also referred to as“tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Pharmaceutically acceptable excipient” or “pharmaceutically acceptable carrier” refers to a component that may be included in the compositions described herein and causes no significant adverse toxicological effects to a subject.

As used in herein, “substantially” or “essentially” means nearly totally or completely, for instance, 95% or greater of a given quantity.

The terms “synergy” or “synergistic” are used to mean that the result of the combination of two or more compounds, components or targeted agents is greater than the sum of each agent together. The terms “synergy” or “synergistic” also means that there is an improvement in the disease condition or disorder being treated, over the use of the two or more compounds, components or targeted agents while each compound, component or targeted agent individually. This improvement in the disease condition or disorder being treated is a “synergistic effect”. A “synergistic amount” is an amount of the combination of the two compounds, components or targeted agents that results in a synergistic effect, as “synergistic” is defined herein. Determining a synergistic interaction between one or two components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods.

The term “pharmaceutically acceptable salt,” as used herein, refers to pharmaceutically acceptable organic or inorganic salts of a compound of the invention. Some embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, trifluoroacetate and xinofoate salts. For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the invention. The materials, methods, and examples are illustrative only and not intended to be limiting.

### Methods and Uses

Provided herein are methods, uses, and combinations, for treating a cancer and/or a cancer-associated disease in a subject that involves combination therapy which comprises at least a first therapeutic agent that is an EZH2 inhibitor, a second therapeutic agent that is a CDK inhibitor, and a third therapeutic agent that is a SERD.

### EZH2 Inhibitors

In some aspects, an EZH2 inhibitor may be selected from, CPI-1205 (Constellation Pharmaceuticals, CAS No.: 1621862-70-1, WO2014124418A1), GSK126 (GlaxoSmithKline, CAS No.: 1346574-57-9), valemetostat (Daiichi Sankyo Company, CAS No.: 1809336-39-7, WO2011140324A1), tazemetostat (Epizyme Inc., CAS No.: 1403254-99-8, WO2012142504A1), PF-06821497 (Pfizer Inc., CAS No.: 1844849-10-0, U.S. Pat. No. 9,481,666 B2), GSK-2816126 (GlaxoSmithKline, CAS No.: 1346574-57-9, WO2011140324A1), 3-deazaneplanocin A (CAS No.: 102052-95-9), or a pharmaceutically acceptable salt thereof.

Preferably EZH2 inhibitor is PF-06821497, which is the compound of Formula A:

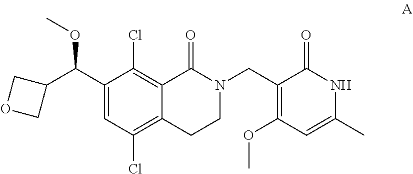 [SMILES: COc1cc(C)[nH]c(=O)c1CN1CCc2c(Cl)cc([C@H](OC)C3COC3)c(Cl)c2C1=O]

or a pharmaceutically acceptable salt thereof.

More preferably, the EZH2 inhibitor as used in the combination therapy is the compound of Formula A.

The compound of Formula A has the chemical name 5,8-dichloro-2-[(4-methoxy-6-methyl-2-oxo-1,2-dihydro-pyridin-3-yl)methyl]-7-[(R)-methoxy(oxetan-3-yl)methyl]-3,4-dihydroisoquinolin-1(2H)-one. The compound of Formula A has been disclosed in the U.S. Pat. No. 9,481,666 B2 and is an exemplary “EZH2 inhibitor”—a class of agents targeting enhancer of zeste homolog 2 (EZH2). The compound of Formula A may be also referred as “PF-06821497”, “PF1497”, or “PF1497” in this disclosure.

### CDK Inhibitors

CDK inhibitors in this disclosure refer to a class of agents targeting cyclin dependent kinase. Preferably, CDK inhibitors in this disclosure refer to a class of agents targeting one or more of the cyclin dependent kinase 2, 4 and 6 (CDK2, CDK4 and CDK6, respectively).

Preferably, CDK inhibitor is the compound of Formula B:

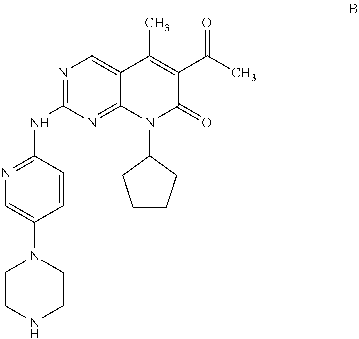 [SMILES: CC(=O)c1c(C)c2cnc(Nc3ccc(N4CCNCC4)cn3)nc2n(C2CCCC2)c1=O]

or a pharmaceutically acceptable salt thereof.

The compound of Formula B is also known as palbociclib, which has the chemical name 6-acetyl-8-cyclopentyl-5-methyl-2-{[5-(piperazin-1-yl)pyridin-2-yl]amino}pyrido[2,3-d]pyrimidin-7(8H)-one. Palbociclib is marketed under the tradename IBRANCE®. Palbociclib is an exemplary “CDK4/6 inhibitor”—a class of agents targeting cyclin dependent kinase 4 and 6 (CDK4 and CDK6, respectively). The compound of Formula B may be also referred as “palbo”, “PD-0332991” or “PD991” in this disclosure.

Palbociclib is described in WHO Drug Information, 2013, Vol. 27, No. 2, page 172. Palbociclib and pharmaceutically acceptable salts thereof, are disclosed in International Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

More preferably, the CDK inhibitor is the compound of Formula C:

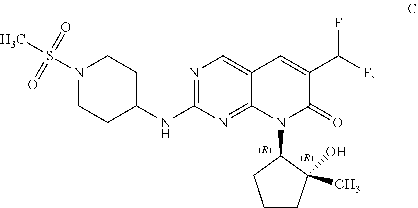

or a pharmaceutically acceptable salt thereof.

The compound of Formula C has the chemical name 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one. The compound of Formula C has been disclosed in the U.S. Pat. No. 10,233,188 B2 and is an exemplary “CDK2/4/6 inhibitor”—a class of agents targeting cyclin dependent kinase 2, 4 and 6 (CDK2, CDK4 and CDK6, respectively). The compound of Formula C may be also referred as “PF-06873600”, “PF-3600”, or “PF3600” in this disclosure.

### Selective Estrogen Receptor Degrader

A selective estrogen receptor degrader or down-regulator (SERD) is a type of drug which binds to the estrogen receptor (ER) and, in the process of doing so, causes the ER to be degraded and thus downregulated.

Preferably, the SERD is fulvestrant or a pharmaceutically acceptable salt thereof. Fulvestrant is a compound of Formula D:

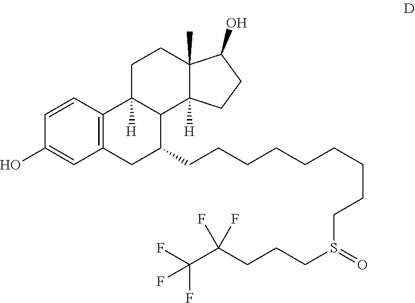

Fulvestrant has the chemical name 7-alpha-[9-(4,4,5,5,5-pentafluoropentylsulphinyl)nonyl]estra-1,3,5-(10)-triene-3,17-beta-diol. Fulvestrant is marketed under the tradename FASLODEX®. The compound of Formula D may be also referred as “Fulv” in this disclosure.

In one embodiment, the present disclosure provides a method of treating a cancer in a subject comprising administering to the subject a combination therapy which comprises a therapeutically effective amount of a first therapeutic agent that is an EZH2 inhibitor, a therapeutically effective amount of a second therapeutic agent that is a CDK inhibitor, and a therapeutically effective amount of a third therapeutic agent that is fulvestrant.

In one preferred embodiment, the present disclosure provides a method of treating a cancer in a subject comprising administering to the subject a combination therapy which comprises a therapeutically effective amount of a first therapeutic agent that is an EZH2 inhibitor selected from CPI-1205 (Constellation Pharmaceuticals), GSK126 (GlaxoSmithKline), valemetostat (Daiichi Sankyo Company), tazemetostat (Epizyme Inc), PF-06821497 (Pfizer Inc.), GSK-2816126 (GlaxoSmithKline), 3-deazaneplanocin A, or a pharmaceutically acceptable salt thereof; a therapeutically effective amount of a second therapeutic agent that is 6-acetyl-8-cyclopentyl-5-methyl-2-{[5-(piperazin-1-yl)pyridin-2-yl]amino}pyrido[2,3-d]pyrimidin-7(8H)-one, or a pharmaceutically acceptable salt thereof, or 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one, or a pharmaceutically acceptable salt thereof; and a therapeutically effective amount of a third therapeutic agent that is fulvestrant, or a pharmaceutically acceptable salt thereof.

In one more preferred embodiment, the present disclosure provides a method of treating a cancer in a subject comprising administering to the subject a combination therapy which comprises a therapeutically effective amount of a first therapeutic agent that is 5,8-dichloro-2-[(4-methoxy-6-methyl-2-oxo-1,2-dihydro-pyridin-3-yl)methyl]-7-[(R)-methoxy(oxetan-3-yl)methyl]-3,4-dihydroisoquinolin-1(2H)-one, or a pharmaceutically acceptable salt thereof; a therapeutically effective amount of a second therapeutic agent that is 6-acetyl-8-cyclopentyl-5-methyl-2-{[5-(piperazin-1-yl)pyridin-2-yl]amino}pyrido[2,3-d]pyrimidin-7(8H)-one, or a pharmaceutically acceptable salt thereof; and a therapeutically effective amount of a third therapeutic agent that is fulvestrant, or a pharmaceutically acceptable salt thereof.

In one even more preferred embodiment, the present disclosure provides a method of treating a cancer in a subject comprising administering to the subject a combination therapy which comprises a therapeutically effective amount of a first therapeutic agent that is 5,8-dichloro-2-[(4-methoxy-6-methyl-2-oxo-1,2-dihydro-pyridin-3-yl)methyl]-7-[(R)-methoxy(oxetan-3-yl)methyl]-3,4-dihydroisoquinolin-1(2H)-one, or a pharmaceutically acceptable salt thereof; a therapeutically effective amount of a second therapeutic agent that is 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one, or a pharmaceutically acceptable salt thereof; and a therapeutically effective amount of a third therapeutic agent that is fulvestrant, or a pharmaceutically acceptable salt thereof.

Preferably, the cancer being treated is breast cancer.

More preferably, the cancer being treated is hormone receptor positive and HER2 negative (HR⁺/HER2⁻) breast cancer.

### Treatment

Each therapeutic agent in a combination therapy of the invention may be administered either alone or in a pharmaceutical composition which comprises the therapeutic agent and one or more pharmaceutically acceptable excipients according to standard pharmaceutical practice.

Each therapeutic agent in a combination therapy of the invention may be administered simultaneously (i.e., in the same pharmaceutical composition), concurrently (i.e., in separate pharmaceutical composition administered one right after the other in any order) in any order.

In some aspects, a therapeutic agents in the combination therapy may be administered using the same dosing regimen (dose, frequency and duration of treatment) that is typically employed when the agent is used as monotherapy for treating the same cancer. In other aspects, the patient may receive a lower total amount of at least one of the therapeutic agents in the combination therapy than when the agent is used as monotherapy, e.g., smaller doses, less frequent doses, and/or shorter treatment duration.

Therapeutic agents in a combination therapy of the invention may be administered by any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient. Examples of parenteral routes of administration also include intraosseous and intrapleural.

Oral administration of a solid dose form of a therapeutic agent may be, for example, presented in discrete units, such as hard or soft capsules, pills, cachets, lozenges, or tablets, each containing a predetermined amount of at least one therapeutic agent. In another aspect, the oral administration may be in a powder or granule form. In another aspect, the oral dose form is sub-lingual, such as, for example, a lozenge. In such solid dosage forms, therapeutic agents are ordinarily combined with one or more adjuvants. Such capsules or tablets may contain a controlled-release formulation. In the case of capsules, tablets, and pills, the dosage forms also may comprise buffering agents or may be prepared with enteric coatings.

In another aspect, oral administration of a therapeutic agent may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In some aspects, therapeutic agents are administered in a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents, and include depot formulations.

In some aspects, therapeutic agents are administered in a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When therapeutic agents are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated-see, for example, Finnin and Morgan, J. Pharm. Sci., 88 (10), 955-958 (1999).

Other carrier materials and modes of administration known in the pharmaceutical art may also be used with therapeutic agents. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3.sup.rd Ed.), American Pharmaceutical Association, Washington, 1999.

Selecting a dosage regimen (also referred to herein as an administration regimen) for a combination therapy of the invention may depend on several factors, including the serum or tissue turnover rate of the entity, the level of symptoms, the immunogenicity of the entity, and the accessibility of the target cells, tissue or organ in the subject being treated. Preferably, a dosage regimen maximizes the amount of each therapeutic agent delivered to the patient consistent with an acceptable level of side effects. Accordingly, the dose amount and dosing frequency of each therapeutic agent or chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

In some aspects, therapeutic agents in a combination therapy of the invention may be administered at least once daily, once a day, twice a day, three times a day, four times a day, once every two days, once every three days, once a week, once every two weeks, once every three weeks, once every four weeks, once every 30 days, once every five weeks, once every six weeks, once a month, once every two months, once every three months, or once every four months in an oral, IV or SC dose.

The treatment methods described herein can continue for as long as the clinician overseeing the patient's care deems the treatment method to be effective. Non-limiting parameters that indicate the treatment method is effective include any one or more of the following: tumor shrinkage (in terms of weight and/or volume); a decrease in the number of individual tumor colonies; tumor elimination; and progression-free survival. Change in tumor size may be determined by any suitable method such as imaging. Various diagnostic imaging modalities well known in the art can be employed, such as computed tomography (CT scan), dual energy CDT, positron emission tomography, ultrasound, CAT scan and MRI. In some aspects, a combination therapy of the invention is used to treat a tumor that is large enough to be found by palpation or by imaging techniques well known in the art, such as MRI, ultrasound, or CAT scan.

The presently described combinations and methods can be used to treat a patient suffering from any condition that can be remedied or prevented by the methods provided herein, such as cancer and/or cancer-associated disease.

In some aspects, the condition is a cancer, including but not limited to, carcinoma, lymphoma, leukemia, myeloma, blastoma, and sarcoma. In some aspects, the cancer is gastric cancer, small intestine cancer, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g., non-small-cell lung cancer, small cell lung cancer), mesothelioma, ovarian cancer, breast cancer, prostate cancer, kidney cancer, esophageal cancer, pancreatic cancer, glioma, renal cancer (e.g., renal cell carcinoma), breast cancer (such as HR+HER2− breast cancer), cervical cancer, uterine cancer, vulvar cancer, endometrial cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, or cancer with high microsatellite instability (MSI-H).

A combination therapy of the invention may be used prior to or following surgery to remove a tumor and may be used prior to, during or after radiation therapy.

In some aspects, a combination therapy of the invention is administered to a patient who has not been previously treated with a therapeutic or chemotherapeutic agent, i.e., is treatment-naïve. In other aspects, the combination therapy is administered to a patient who failed to achieve a sustained response after prior therapy with a therapeutic or chemotherapeutic agent, i.e., is treatment-experienced. In some aspects, the subject has received a prior therapy to treat the tumor and the tumor is relapsed or refractory.

Encompassed by the invention provided herein are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain aspects, the methods and compositions provided herein permit treatment or prevention of diseases and disorders wherein treatment is improved by an enhanced anti-tumor response using lower and/or less frequent doses of at least therapeutic agent in a combination therapy to at least one of i) reduce the incidence of unwanted or adverse effects caused by the administration of the therapeutic agents separately, while at least maintaining efficacy of treatment; ii) increase patient compliance, and iii) improve efficacy of the anti-tumor treatment.

### Embodiments (EBs)

EB1. A method of treating a cancer in a subject comprising administering to the subject a combination therapy which comprises a therapeutically effective amount of a first therapeutic agent that is an EZH2 inhibitor or a pharmaceutically acceptable salt thereof; a therapeutically effective amount of a second therapeutic agent that is a CDK inhibitor or a pharmaceutically acceptable salt thereof; and a therapeutically effective amount of a third therapeutic agent that is fulvestrant or a pharmaceutically acceptable salt thereof.

EB2. The method of EB1, wherein the EZH2 inhibitor is CPI-1205, GSK126, valemetostat, tazemetostat, PF-06821497, GSK-2816126, 3-deazaneplanocin A, or a pharmaceutically acceptable salt thereof.

EB3. The method of EB1 or EB2, wherein the EZH2 inhibitor is PF-06821497 having Formula A:

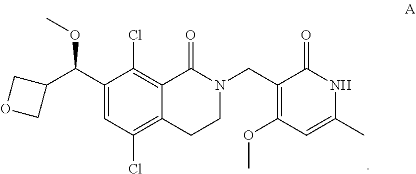 [SMILES: COc1cc(C)[nH]c(=O)c1CN1CCc2c(Cl)cc([C@H](OC)C3COC3)c(Cl)c2C1=O]

EB4. The method of any one of cEB1 to EB3, wherein the CDK inhibitor is palbociclib having Formula B:

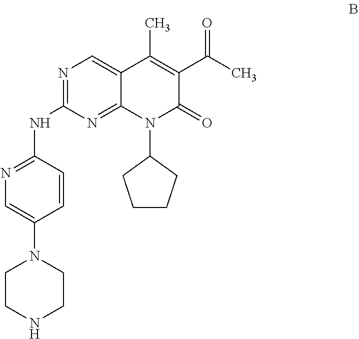 [SMILES: CC(=O)c1c(C)c2cnc(Nc3ccc(N4CCNCC4)cn3)nc2n(C2CCCC2)c1=O]

or a pharmaceutically acceptable salt thereof.

EB5. The method of any one of EB1 to EB3, wherein the CDK inhibitor is PF-06873600 having Formula C:

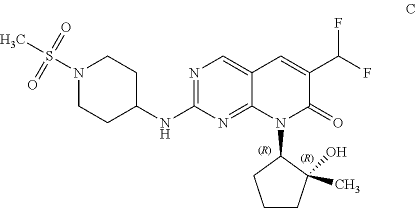

or a pharmaceutically acceptable salt thereof.

EB6. The method of any one EB1 to EB3, wherein the EZH2 inhibitor is PF-06821497; and the CDK inhibitor is palbociclib or a pharmaceutically acceptable salt thereof.

EB7. The method of any one of EB1 to EB3, wherein the EZH2 inhibitor is PF-06821497; and the CDK inhibitor is PF-06873600 or a pharmaceutically acceptable salt thereof.

EB8. The method of any one of EB1 to EB7, wherein the cancer is breast cancer.

EB9. The method of any one of EB1 to EB8, wherein the breast cancer is hormone receptor (HR) positive and human epidermal growth factor 2 (HER2) negative breast cancer.

EB10. The method of one any of EB1 to EB9, wherein each therapeutic agent is administered simultaneously or separately.

EB11. A combination therapy, wherein the combination therapy comprises an EZH2 inhibitor that is CPI-1205, GSK126, valemetostat, tazemetostat, PF-06821497, GSK-2816126, 3-deazaneplanocin A, or a pharmaceutically acceptable salt thereof; a CDK inhibitor that is palbociclib or a pharmaceutically acceptable salt thereof, or PF-06873600 or a pharmaceutically acceptable salt thereof; and fulvestrant or a pharmaceutically acceptable salt thereof.

EB12. The combination therapy of EB11, wherein the combination therapy comprises PF-06821497, palbociclib or a pharmaceutically acceptable salt thereof, and fulvestrant.

EB13. The combination therapy of EB11, wherein the combination therapy comprises PF-06821497, PF-06873600 or a pharmaceutically acceptable salt thereof, and fulvestrant.

EB14. The combination therapy of any one of EB11 to EB13, for use in the manufacture of a medicament for treating cancer.

EB15. The combination therapy of EB14, for use in the manufacture of a medicament for treating breast cancer.

EB16. The combination therapy of EB15, for use in the manufacture of a medicament for treating HR positive and human HER2 negative breast cancer.

EB17. The combination therapy of any one of EB11 to EB13, for use in the treatment of cancer.

EB18. The combination therapy for use in the treatment of cancer of EB17, wherein the cancer is breast cancer.

EB19. The combination therapy for use in the treatment of cancer of EB18, wherein the breast cancer is HR positive and HER2 negative breast cancer.

### EXAMPLES

In order that this invention may be better understood, the following examples are set forth. These examples are for purposes of illustration only and are not to be construed as limiting the scope of the invention in any manner.

The purpose of examples was to assess whether an EZH2 inhibitor such as PF-06821497 can improve the benefit of a CDK inhibitor plus fulvestrant therapy in HR+HER2− breast cancer models. The efficacy of triple-agent treatment with PF-06821497, Palbociclib or PF-06873600, and fulvestrant was evaluated in the HCC1428 breast cancer model and the ST941 PDX breast cancer model. Additionally, the triple combination of PF-06821497, PF-06873600 (CDK2/4/6 inhibitor) plus fulvestrant was assessed in the ST941 PDX model.

### Example 1: In Vivo Efficacy Evaluation of Triple Agent Combination with a) PF-06821497, Palbociclib and Fulvestrant, or b) PF-06821497, PF-06873600 and Fulvestrant in the HR+/HER2− HCC1428 Breast Cancer Model

### Materials and Methods

HCC1428 cells (ATCC, CRL-2327) were maintained in PRMI-1640 medium, plus 10% FBS until ready for implant. HCC1428 model was established by serial in vivo propagation. To establish the HCC1428 donor mice, tumor cells (5×10⁶ cell/mouse with 50% Cultrex® Basement Membrane Matrix) were subcutaneously implanted in female NSG mice with estrogen supplementation (SC implanted, in 0.36 mg 90-day release, Innovative Research of America, cat #NE-121). Once reaching a range of 700 to 800 mm3, donor tumors were subsequently transplanted into secondary recipient mice for a study expansion. In this study, the HCC1428 model was established by implanting passage 4 tumor fragments into recipient mice. When the mean tumor volume reached around 119 mm3, the tumor bearing mice were randomly assigned into 6 groups (n=10 per group) and dosed with 1) vehicle (2.5% PVP K30/0.5% Pluronic F127/Water), sc; 2) 100 mg/kg PF-06821497, sc; 3) 10 mg/kg palbociclib (in 0.5% MC with 0.1% Tween 80 in water, po), plus fulvestrant (in 100% peanut oil, sc); 4) 25 mg/kg PF-06873600 (in 0.5% MC with 0.1% Tween 80 in water); 5) PF-06821497, palbociclib and fulvestrant triple-agent combination; and 6) PF-06821497, PF-06873600 and fulvestrant triple-agent combination. Palbociclib and PF-06873600 was dosed twice daily (7 hr apart). PF-06821497 and vehicle was dosed once daily. Fulvestrant was administered twice in the first week, then weekly afterwards. TGI was assessed on Day 36 post first dose.

### Efficacy Evaluation and Statistical Analysis

Tumor volume and body weights were measured twice a week after dosing initiation. Tumor volume i was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. All mice received treatment continuously until the day of efficacy assessment. Statistical analysis was performed in GraphPad Prism with an unpaired t test, followed by Mann-Whitney test.

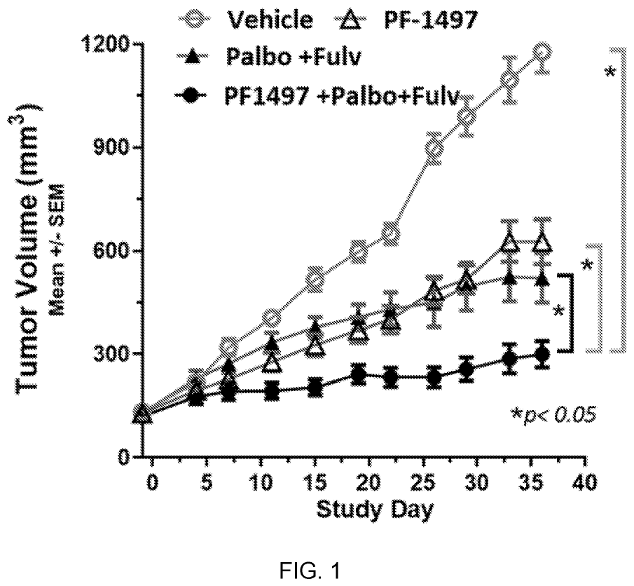

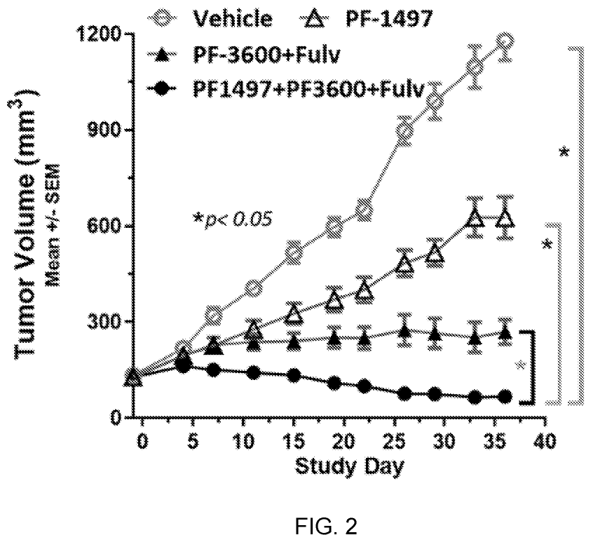

The HCC1428 tumor bearing mice were randomized into 6 groups when the mean tumor volume reached around 119 mm3 and subsequently treated. PF-06821497 (100 mg/kg QD), PF-06873600 (25 mg/kg BID)/fulvestrant (10 mg/kg) doublet or palbociclib (10 mg/kg BID)/fulvestrant doublet exhibited significant antitumor efficacy vs vehicle (p<0.05) respectively. Cotreatment with PF-06821497 significantly enhanced the benefit of palbociclib/fulvestrant doublet (p<0.05) or PF-06873600/fulvestrant doublet (p<0.05), respectively. In addition, the triplet treatments also demonstrated significantly improved efficacy vs PF-06821497 monotherapy (p<0.05). See FIG. 1, FIG. 2, and Table 1.

TABLE 1
Efficacy evaluation of PF-06821497 single or
combination treatment in the HCC1428 model

| | | | | P- | |
|---|---|---|---|---|---|
| | | | % | value | |
| | Dose | | TGI | vs | |
| Group | mg/kg | Schedule | (D36) | veh | N |
| Vehicle | — | SC (QD) | — | — | 10 |
| PF-06821497 | 100 | SC (QD) | 55 | <0.05 | 10 |
| Palbociclib + | 10 + | PO (BID)/ | 66 | <0.05 | 10 |
| fulvestrant | 10 | SC (D1, 3, 8 | | | |
| | | then weekly) | | | |
| PF-06873600 + | 25 + | PO (BID)/ | 89 | <0.05 | 10 |
| fulvestrant | 10 | SC (D1, 3, 8 | | | |
| | | then weekly) | | | |
| PF-06821497 + | 100 + | SC (QD)/ | 85^{♦a} | <0.05 | 10 |
| Palbociclib + | 10 + | PO (BID)/ | | | |
| fulvestrant | 10 | SC (D1, 3, 8 | | | |
| | | then weekly) | | | |
| PF-06821497 + PF- | 100 + | SC (QD)/PO | 1080^{∞a} | <0.05 | 10 |
| 06873600 + | 25 + | (BID)/ | | | |
| fulvestrant | 10 | SC (D1, 3, 8 | | | |
| | | then weekly) | | | |

Statistical analysis was performed in GraphPad Prism with an unpaired t test, followed by Mann-Whitney test.
^{♦}indicates p < 0.05 vs. Palbociclib + fulvestrant treatment;
^{∞}indicates p < 0.05 vs PF-06873600 + fulvestrant;
^{a}indicates p < 0.05 vs PF-06821497.

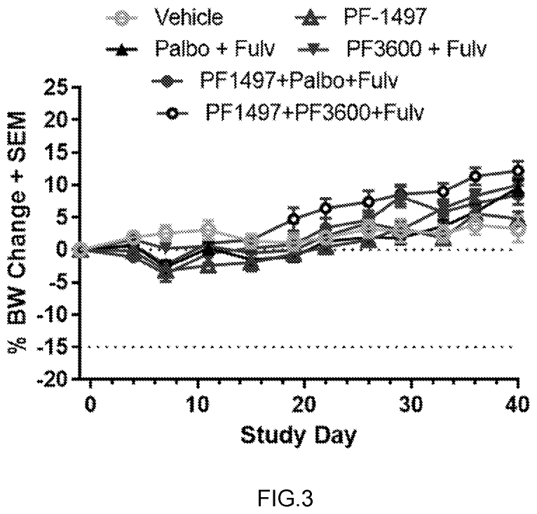

The body weight change during the treatment can be found in FIG. 3. The body weight of experimental animals is used as a reference index for indirect determination of drug toxicity. In this model, none of the administration groups showed weight loss at the end of dosing period.

Compared to the dual-combination therapy as disclosed in WO 2021/063322 A1, the triple-agent combination therapy of the present disclosure provides extended antitumor efficacy (consistent tumor size decrease over at least a 35-day period as seen in Table 1 and FIG. 2, for the combination therapy of PF-06821497, PF-06873600, and fulvestrant) and/or better toxicity profile (no body weight loss as seen in FIG. 3).

### Example 2: In Vivo Efficacy Evaluation of Triple Agent Combination with PF-06821497, Palbociclib and Fulvestrant in the HR+/HER2− ST941 PDX Model

### Materials and Methods

The HR+/HER2− PDX studies were performed at START (San Antonio, TX) using female athymic nude mice (Foxn1nu/Foxn1nu, JAX Laboratories). Mice received 8.5 μg/mL β estradiol drinking water during study. The live donor mice were established by implanting cryopreserved tumor fragments (around 70 mg). Once ready, the donor tumor fragments were subsequently transplanted into secondary recipient mice for a study expansion. For efficacy evaluation, the tumor bearing mice were randomly assigned to groups when the mean tumor volumes reached around 177 mm3, and subsequently treated with: 1) vehicle (2.5% PVP K30/0.5% Pluronic F127/Water), sc; 2) 100 mg/kg PF-06821497, sc; 3) 10 mg/kg palbociclib (in 0.5% MC with 0.1% Tween 80 in water, po), plus fulvestrant (in 100% peanut oil, sc); 4) PF-06821497, palbocidib and fulvestrant triple-agent combination.

Palbocidib was dosed twice daily (7 hr apart). PF-06821497 and vehicle was dosed once daily. Fulvestrant was administered twice in the first week, then weekly afterwards. TGI was assessed on Day 36 post first dose.

### Efficacy Evaluation and Statistical Analysis

Tumor volume and body weights were measured twice a week after dosing initiation. Tumor volume i was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. All mice received treatment continuously until the day of efficacy assessment. Statistical analysis was performed in GraphPad Prism with an unpaired t test, followed by Mann-Whitney test.

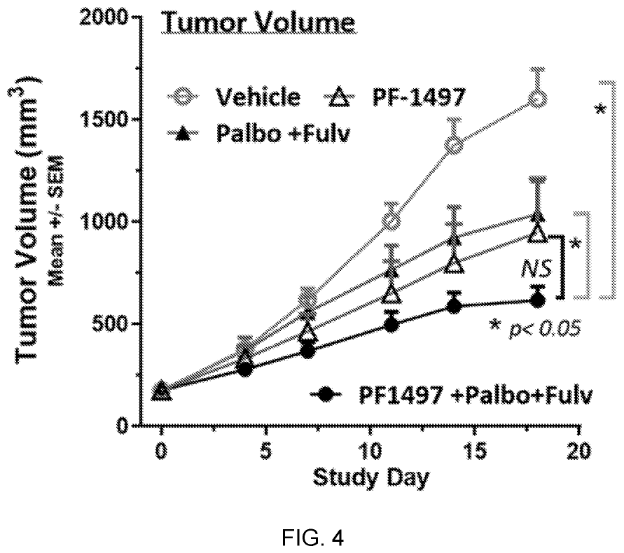

The ST941 tumor bearing mice were randomized into 4 groups when the mean tumor volume reached around 177 mm3 and subsequently treated. PF-06821497 (100 mg/kg QD) or palbociclib (10 mg/kg BID) plus fulvestrant (10 mg/kg) doublet exhibited significant antitumor efficacy vs vehicle (p<0.05) respectively. Co-treatment with PF-06821497 significantly enhanced the benefit of palbocidib/fulvestrant doublet (p<0.05). See FIG. 4, and Table 2.

TABLE 2
Efficacy evaluation of PF-06821497 single or
combination treatment in the ST941 PDX model

| | Dose | | % TGI | P-value | |
|---|---|---|---|---|---|
| Group | mg/kg | Schedule | (D18) | vs veh | N |
| Vehicle | — | SC (QD) | — | — | 10 |
| PF-06821497 | 100 | SC (QD) | 44.6% | <0.05 | 10 |
| Palbociclib + | 10 + 10 | PO (BID)/ | 39.4% | <0.05 | 10 |
| fulvestrant | | SC (D1, 3, 8 | | | |
| | | then weekly) | | | |
| PF-06821497 + | 100 + | SC (QD)/ | 66.8%^{♦} | <0.05 | 10 |
| Palbociclib + | 10 + | PO (BID)/ | | | |
| fulvestrant | 10 | SC (D1, 3, 8 | | | |
| | | then weekly) | | | |

Statistical analysis was performed in GraphPad Prism with an unpaired t test, followed by Mann-Whitney test.
^{♦}indicates p < 0.05 vs. Palbociclib + fulvestrant treatment.

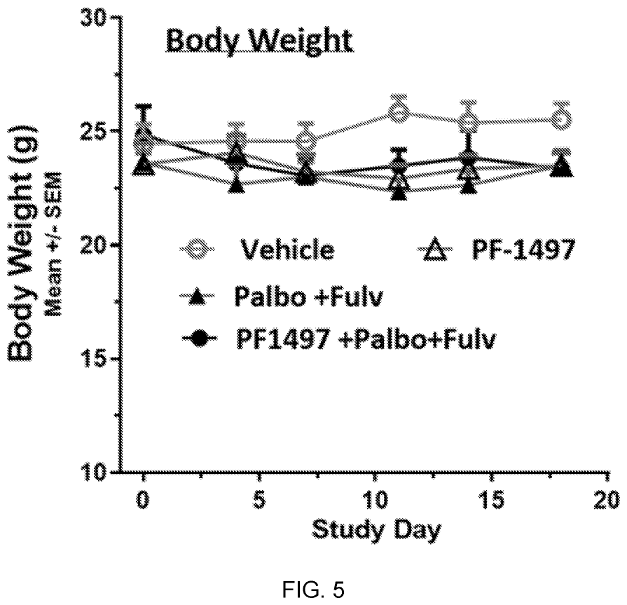

The body weight change during the treatment can be found in FIG. 5. The body weight of experimental animals is used as a reference index for indirect determination of drug toxicity. In this model, none of the administration groups showed significant weight losses.

PF-06821497 in combination with palbociclib/fulvestrant doublet resulted in significant (p<0.05) combinatorial benefit vs single agent PF-06821497 or palbociclib/fulvestrant doublet in the HCC1428 model. The triplet combination therapy of PF-06821497, PF-06873600 and fulvestrant demonstrated even more significantly (p<0.05) improved efficacy vs PF-06821497 or PF-06873600/fulvestrant doublet. The combinatorial benefit was also observed in the ST941 PDX model. PF-06821497 in combination with palbociclib/fulvestrant significantly (p<0.05) improved efficacy vs palbociclib/fulvestrant doublet.

The foregoing description and Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

1. A method of treating a cancer in a subject comprising administering to the subject a combination therapy which comprises a therapeutically effective amount of a first therapeutic agent that is an EZH2 inhibitor or a pharmaceutically acceptable salt thereof; a therapeutically effective amount of a second therapeutic agent that is a CDK inhibitor or a pharmaceutically acceptable salt thereof; and a therapeutically effective amount of a third therapeutic agent that is fulvestrant or a pharmaceutically acceptable salt thereof.

2. The method of claim 1, wherein the EZH2 inhibitor is CPI-1205, GSK126, valemetostat, tazemetostat, PF-06821497, GSK-2816126, 3-deazaneplanocin A, or a pharmaceutically acceptable salt thereof.

3. The method of claim 1, wherein the EZH2 inhibitor is PF-06821497 having Formula A:

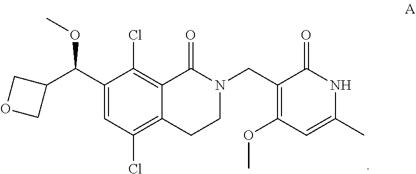 [SMILES: COc1cc(C)[nH]c(=O)c1CN1CCc2c(Cl)cc([C@H](OC)C3COC3)c(Cl)c2C1=O]

4. The method of claim 1, wherein the CDK inhibitor is palbociclib having Formula B: 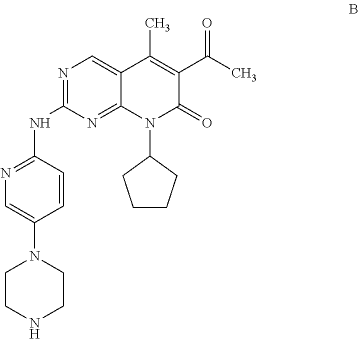 [SMILES: CC(=O)c1c(C)c2cnc(Nc3ccc(N4CCNCC4)cn3)nc2n(C2CCCC2)c1=O]
or a pharmaceutically acceptable salt thereof.

5. The method of claim 1, wherein the CDK inhibitor is PF-06873600 having Formula C: 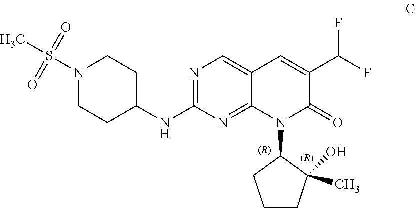
or a pharmaceutically acceptable salt thereof.

6. The method of claim 1, wherein the EZH2 inhibitor is PF-06821497; and the CDK inhibitor is palbociclib or a pharmaceutically acceptable salt thereof.

7. The method of claim 1, wherein the EZH2 inhibitor is PF-06821497; and the CDK inhibitor is PF-06873600 or a pharmaceutically acceptable salt thereof.

8. The method of claim 1, wherein the cancer is breast cancer.

9. The method of claim 8, wherein the breast cancer is hormone receptor (HR) positive and human epidermal growth factor 2 (HER2) negative breast cancer.

10. The method of claim 1, wherein each therapeutic agent is administered simultaneously or separately.

11. A combination therapy, wherein the combination therapy comprises an EZH2 inhibitor that is CPI-1205, GSK126, valemetostat, tazemetostat, PF-06821497, GSK-2816126, 3-deazaneplanocin A, or a pharmaceutically acceptable salt thereof; a CDK inhibitor that is palbociclib or a pharmaceutically acceptable salt thereof, or PF-06873600 or a pharmaceutically acceptable salt thereof; and fulvestrant or a pharmaceutically acceptable salt thereof.

12. The combination therapy of claim 11, wherein the combination therapy comprises PF-06821497, palbociclib or a pharmaceutically acceptable salt thereof, and fulvestrant.

13. The combination therapy of claim 11, wherein the combination therapy comprises PF-06821497; PF-06873600 or a pharmaceutically acceptable salt thereof; and fulvestrant.

14. The combination therapy of claim 11, for use in the manufacture of a medicament for treating cancer.

15. The combination therapy of claim 14, for use in the manufacture of a medicament for treating breast cancer.

16. The combination therapy of claim 15, for use in the manufacture of a medicament for treating HR positive and human HER2 negative breast cancer.

17. The combination therapy of claim 11, for use in the treatment of cancer.

18. The combination therapy for use in the treatment of cancer of claim 17, wherein the cancer is breast cancer.

19. The combination therapy for use in the treatment of cancer of claim 18, wherein the breast cancer is HR positive and HER2 negative breast cancer.
